(19) United States
(12) Patent Application Publication
(10) Pub. No.: US 2011/0136247 A1
(43) Pub. Date: Jun. 9, 2011

(54) PHOTOLUMINESCENT OXYGEN PROBE WITH REDUCED CROSS-SENSITIVITY TO HUMIDITY

(76) Inventors: Dmitri Boris Papkovsky, County Cork (IE); Daniel W. Mayer, Wyoming, MN (US)

(21) Appl. No.: 12/632,318

(22) Filed: Dec. 7, 2009

(51) Int. Cl. G01N 33/00; G01N 21/64; B05D 3/02; B05D 5/10

(52) U.S. Cl. 436/136; 422/82.08; 427/385.5; 427/208.8

(57) ABSTRACT

An oxygen-sensitive probe having reduced cross-sensitivity to humidity and methods of manufacturing and using such probes to measure oxygen concentrations within an enclosed space. The probe includes a thin film of an oxygen-sensitive photoluminescent dye on a first major surface of a microporous wettable polyolefin support layer. The dye is preferably a solid state composition comprising the oxygen-sensitive photoluminescent dye embedded within an oxygen-permeable hydrophobic polymer matrix.

### BACKGROUND

Solid-state polymeric materials based on oxygen-sensitive photoluminescent dyes are widely used as optical oxygen sensors and probes. See, for example United States Published Patent Applications 2009/0029402, 2008/8242870, 2008/215254, 2008/199360, 2008/190172, 2008/148817, 2008/146460, 2008/117418, 2008/0051646, 2006/0002822, U.S. Pat. Nos. 7,569,395, 7,534,615, 7,368,153, 7,138,270, 6,689,438, 5,718,842, 4,810,655, and 4,476,870. Such optical sensors are available from a number of suppliers, including Presens Precision Sensing, GmbH of Regensburg, Germany, Oxysense of Dallas, Tex., United States, and Luxcel Biosciences, Ltd of Cork, Ireland.

To increase photoluminescent signals obtainable from the sensor and thus increase the reliability of optical measurements, oxygen-sensitive materials often incorporate a light-scattering additive (e.g., TiO2—-Klimant I., Wolfbeis O. S.—Anal Chem, 1995, v.67, p. 3160-3166) or underlayer (e.g., microporous support—see Papkovsky, D B et al.—Sensors Actuators B, 1998, v.51, p. 137-145). Unfortunately, such probes tend to show significant cross-sensitivity to humidity, preventing them from gaining wide acceptance for use in situations where humidity of the samples under investigation cannot be controlled.

Hence, a need exists for an optical photoluminescent oxygen probe with reduced cross-sensitivity to humidity.

### SUMMARY OF THE INVENTION

A first aspect of the invention is an oxygen-sensitive probe comprising an oxygen-sensitive photoluminescent dye applied onto a first major surface of a microporous wettable polyolefin support layer so as to form a thin film of the photoluminescent dye on the support layer. The oxygen-sensitive photoluminescent dye is preferably applied as a solid state composition comprising the oxygen-sensitive photoluminescent dye embedded within an oxygen-permeable polymer matrix.

A second aspect of the invention is a method for measuring oxygen concentration within an enclosed space employing an oxygen-sensitive probe according to the first aspect of the invention. The method includes the steps of (A) obtaining an oxygen-sensitive probe according to the first aspect of the invention, (B) placing the probe within the enclosed space, and (C) ascertaining oxygen concentration within the enclosed space by (i) repeatedly exposing the probe to excitation radiation over time, (ii) measuring radiation emitted by the excited probe after at least some of the exposures, (iii) measuring passage of time during the repeated excitation exposures and emission measurements, and (iv) converting at least some of the measured emissions to an oxygen concentration based upon a known conversion algorithm.

A third aspect of the invention is a method for monitoring changes in oxygen concentration within an enclosed space employing an oxygen-sensitive probe according to the first aspect of the invention. The method includes the steps of (A) obtaining an oxygen-sensitive probe according to the first aspect of the invention, (B) placing the probe within the enclosed space, (C) ascertaining oxygen concentration within the enclosed space over time by (i) repeatedly exposing the probe to excitation radiation over time, (ii) measuring radiation emitted by the excited probe after at least some of the exposures, (iii) measuring passage of time during the repeated excitation exposures and emission measurements, and (iv) converting at least some of the measured emissions to an oxygen concentration based upon a known conversion algorithm, and (D) reporting at least one of (i) at least two ascertained oxygen concentrations and the time interval between those reported concentrations, and (ii) a rate of change in oxygen concentration within the enclosed space calculated from data obtained in step (C).

A fourth aspect of the invention is a method of preparing a photoluminescent oxygen-sensitive probe according to the first aspect of the invention. The method includes the steps of (A) preparing a coating cocktail which contains the photoluminescent oxygen-sensitive dye and the oxygen-permeable polymer in an organic solvent, (B) applying the cocktail to the first major surface of the support material, and (C) allowing the cocktail to dry, whereby a solid-state thin film coating is formed on the support, thereby forming the photoluminescent oxygen-sensitive probe.

### BRIEF DESCRIPTION OF THE DRAWINGS

FIG. 1 is a grossly enlarge cross-sectional side view of a central portion of one embodiment of the invention.

### DETAILED DESCRIPTION OF A PREFERRED EMBODIMENT

### Definitions

As used herein, including the claims, the phrase “oxygen permeable” means a material that when formed into a 1 mil film has an oxygen transmission rate of greater than 1,000 c³/m2 day when measured in accordance with ASTM D 3985.

As used herein, including the claims, the term “spinlaid” means a process for producing fibrous nonwoven fabric directly from extruded polymer fibers and includes spunbond and meltblown techniques.

As used herein, including the claims, the phrase “thin film” means a film having a thickness of less than 10 μm.

As used herein, including the claims, the term “wettable” means the ability of water to maintain contact with the surface of the solid sufficient to provide good aqueous wicking characteristics.

As used herein, including the claims, the phrase “moderately wettable” means that water maintains a contact angle θ of less than 90°.

As used herein, including the claims, the phrase “highly wettable” means that water maintains a contact angle θ of less than 60°.

As used herein, including the claims, the phrase “completely wettable” means that water maintains a contact angle θ of less than 30°.

### Nomenclature

10 Probe
20 Solid State Composition
21 Oxygen-Sensitive Photoluminescent Dye
22 Oxygen-Permeable Polymer Matrix
30 Support Layer
30a First or Upper Major Surface of Support Layer
30b Second or Lower Major Surface of Support Layer
40 Pressure Sensitive Adhesive Layer

### Description

Construction

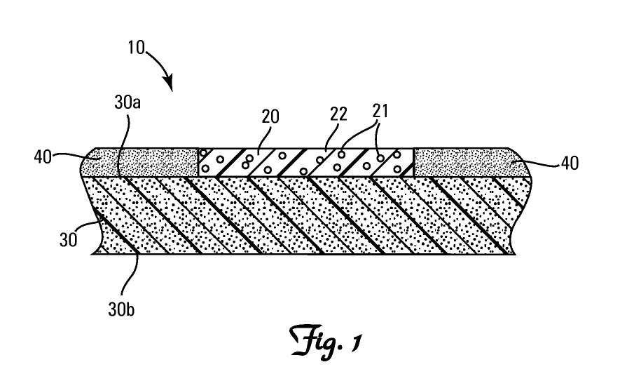

Referring generally to FIG. 1, a first aspect of the invention is an oxygen-sensitive probe or sensor 10 useful for optically measuring oxygen concentration within an enclosed space (not shown), such as the retention chamber (not shown) of a hermetically sealed package (not shown). The probe 10 includes a thin film of a solid state photoluminescent composition 20 coated onto a first major surface 30a of a support layer 30. The solid state photoluminescent composition 20 includes an oxygen-sensitive photoluminescent dye 21 embedded within an oxygen-permeable polymer matrix 22.

The oxygen-sensitive photoluminescent dye 21 used in the solid state photoluminescent composition 20 may be selected from any of the well-known oxygen sensitive photoluminescent dyes 21. One of routine skill in the art is capable of selecting a suitable dye 21 based upon the intended use of the probe 10. A nonexhaustive list of suitable oxygen sensitive photoluminescent dyes 21 includes specifically, but not exclusively, ruthenium(II)-bipyridyl and ruthenium(II)-diphenylphenanothroline complexes, porphyrin-ketones such as platinum(II)-octaethylporphine-ketone, platinum(II)-porphyrin such as platinum(II)-tetrakis(pentafluorophenyl)porphine, palladium(II)-porphyrin such as palladium(II)-tetrakis(pentafluorophenyl)porphine, phosphorescent metallocomplexes of tetrabenzoporphyrins, chlorins, azaporphyrins, and long-decay luminescent complexes of iridium(III) or osmium(II).

Typically, the hydrophobic oxygen-sensitive photoluminescent dye 21 is compounded with a suitable oxygen-permeable and hydrophobic carrier matrix 22. Again, one of routine skill in the art is capable of selecting a suitable oxygen-permeable hydrophobic carrier matrix 22 based upon the intended use of the probe 10 and the selected dye 21. A nonexhaustive list of suitable polymers for use as the oxygen-permeable hydrophobic carrier matrix 22 includes specifically, but not exclusively, polystryrene, polycarbonate, polysulfone, polyvinyl chloride and some co-polymers.

Referring again to FIG. 1, the probe 10 preferably includes a layer of a pressure sensitive adhesive 40 on the first major surface 30a of the support layer 30 for facilitating attachment of the probe 10 to a surface (not shown) of a container (not shown) that defines the enclosed space (not shown) whose oxygen concentration is to be measured, with the photoluminescent solid state composition 20 on the probe 10 facing outward from the container (not shown) through an area of the container (not shown) that is transparent or translucent to radiation at the excitation and emission wavelengths of the dye 21 in the photoluminescent solid state composition 20. The adhesive 40 may but should not cover the photoluminescent solid state composition 20.

The support layer 30 is a sheet of a microporous wettable polyolefin with first and second major surfaces 30a and 30b. Such materials, when employed as the support layer 30 for the photoluminescent solid state composition 20, substantially reduces cross-sensitivity of the photoluminescent solid state composition 20 to humidity relative to probes 10 employing other traditional materials. The support layer 30 is preferably highly wettable and most preferably completely wettable. Preferred materials for use as the support layer 30 are non-woven spinlaid fibrous polyolefin fabrics, such as a spunbond polypropylene fabric grafted with hydrophilic pendant groups such as acrylic acid. One such fabric is available from Freudenberg Nonwovens NA of Hopkinsville, Ky. and Freudenberg Nonwovens Ltd of West Yorkshire, United Kingdom under the designation 700/70 (a nonwoven microporous spunbond polypropylene fabric grafted with acrylic acid to render the polymer wettable and etched with a caustic). In addition, this type of support material substantially increases the luminescent intensity signals obtainable from the sensor 10 and improves mechanical properties of the oxygen-sensitive coating (when compared to traditional sensors on planar, non-porous polymeric support such as polyester Mylar® film).

The support layer 30 is preferably between about 30 μm and 500 μm thick.

The probes 10 of the present invention have little cross-sensitivity to humidity, with a change of luminescence lifetime of less than 5% with a change in relative humidity of an analyte gas from 1% to 90%. By proper selection of the support layer 30, based upon various factors including the particular photoluminescent solid state composition 20 employed, a change in luminescence lifetime of less than 3% and even less than 1% can be readily achieved.

Manufacture

The probe 10 can be manufactured by the traditional methods employed for manufacturing such probes 10. Briefly, the probe 10 can be conveniently manufactured by (A) preparing a coating cocktail (not shown) which contains the photoluminescent oxygen-sensitive dye 21 and the oxygen-permeable polymer 22 in an organic solvent (not shown) such as ethylacetate, (B) applying the cocktail to the first major surface 30a of a support material 30 or soaking the support material in the cocktail (not shown), and (C) allowing the cocktail (not shown) to dry, whereby a solid-state thin film coating 20 is formed on the support 30, thereby forming the photoluminescent oxygen-sensitive probe 10. The resultant probe 10 is preferably heat treated to remove mechanical stress from the sensor material which is associated with its preparation (solidification and substantial volume reduction).

Generally, the concentration of the polymer 22 in the organic solvent (not shown) should be in the range of 0.1 to 20% w/w, with the ratio of dye 21 to polymer 22 in the range of 1:50 to 1:5,000 w/w.

A layer of pressure sensitive adhesive 40 can optionally be coated onto the first major surface 30a of the support material 30 by conventional coating techniques.

Use

The probe 10 can be used to quickly, easily, accurately and reliably measure oxygen concentration within an enclosed space (not shown) regardless of the relative humidity within the enclosed space (not shown). The probe 10 can be used to measure oxygen concentration in the same manner as other oxygen sensitive photoluminescent probes. Briefly, the probe 10 is used to measure oxygen concentration within an enclosed space (not shown) by (A) placing the probe 10 within the enclosed space (not shown) at a location where radiation at the excitation and emission wavelengths of the dye 21 can be transmitted to and received from the photoluminescent solid state composition 20 with minimal interference and without opening or otherwise breaching the integrity of the enclosure, and (B) ascertaining the oxygen concentration within the enclosed space (not shown) by (i) repeatedly exposing the probe 10 to excitation radiation over time, (ii) measuring radiation emitted by the excited probe 10 after at least some of the exposures, (iii) measuring passage of time during the repeated excitation exposures and emission measurements, and (iv) converting at least some of the measured emissions to an oxygen concentration based upon a known conversion algorithm. Such conversion algorithms are well know to and readily developable by those with routine skill in the art.

In a similar fashion, the probe 10 can be used to quickly, easily, accurately and reliably monitor changes in oxygen concentration within an enclosed space (not shown) regardless of the relative humidity within the enclosed space (not shown). The probe 10 can be used to monitor changes in oxygen concentration in the same manner as other oxygen sensitive photoluminescent probes. Briefly, the probe 10 is used to monitor changes in oxygen concentration within an enclosed space (not shown) by (A) placing the probe 10 within the enclosed space (not shown) at a location where radiation at the excitation and emission wavelengths of the dye 21 can be transmitted to and received from the photoluminescent solid state composition 20 with minimal interference and without opening or otherwise breaching the integrity of the enclosure, (B) ascertaining the oxygen concentration within the enclosed space (not shown) over time by (i) repeatedly exposing the probe 10 to excitation radiation over time, (ii) measuring radiation emitted by the excited probe 10 after at least some of the exposures, (iii) measuring passage of time during the repeated excitation exposures and emission measurements, and (iv) converting at least some of the measured emissions to an oxygen concentration based upon a known conversion algorithm, and (C) reporting at least one of (i) at least two ascertained oxygen concentrations and the time interval between those reported concentrations, and (ii) a rate of change in oxygen concentration within the enclosed space calculated from data obtained in step (B). Conversion algorithms used to convert the measured emissions to an oxygen concentration are well know to and readily developable by those with routine skill in the art.

The radiation emitted by the excited probe 10 can be measured in terms of intensity and/or lifetime (rate of decay, phase shift or anisotropy), with measurement of lifetime generally preferred as a more accurate and reliable measurement technique when seeking to establish oxygen concentration via measurement of the extent to which the dye 21 has been quenched by oxygen.

1. An oxygen-sensitive probe comprising a thin film coating of an oxygen-sensitive photoluminescent dye on a first major surface of a microporous wettable polyolefin support layer.

2. The oxygen-sensitive probe of claim 1 further comprising a layer of a pressure-sensitive adhesive applied onto the first major surface of the support layer.

3. The oxygen-sensitive probe of claim 1 wherein the oxygen-sensitive photoluminescent dye is applied as a solid state composition comprising the oxygen-sensitive photoluminescent dye embedded within an oxygen-permeable hydrophobic polymer matrix.

4. The oxygen-sensitive probe of claim 3 wherein the oxygen-sensitive photoluminescent dye is a transition metal complex.

5. The oxygen-sensitive probe of claim 4 wherein the transition metal complex is selected from the group consisting of a ruthenium bipyridyl, a ruthenium diphenylphenanothroline, a platinum porphyrin, a palladium porphyrin, a phosphorescent metallocomplex of a porphyrin-ketone, an azaporphyrin, a tetrabenzoporphyrin, a chlorin, and a long-decay luminescent complex of iridium(III) or osmium(II).

6. The oxygen-sensitive probe of claim 3 wherein the oxygen-permeable polymer matrix is selected from the group consisting of polystryrene, polycarbonate, polysulfone, and polyvinyl chloride.

7. The oxygen-sensitive probe of claim 1 wherein the wettable polyolefin support layer is highly wettable.

8. The oxygen-sensitive probe of claim 1 wherein the wettable polyolefin support layer is completely wettable.

9. The oxygen-sensitive probe of claim 1 wherein the wettable polyolefin support layer is a non-woven spinlaid fibrous polyolefin fabric.

10. The oxygen-sensitive probe of claim 9 wherein the wettable polyolefin support layer is a non-woven spunbond polyolefin fabric.

11. The oxygen-sensitive probe of claim 1 wherein the wettable polyolefin is a wettable polyethylene or polypropylene.

12. The oxygen-sensitive probe of claim 1 wherein the wettable polyolefin support layer is a wettable non-woven spunbond polypropylene fabric.

13. The oxygen-sensitive probe of claim 1 wherein the wettable polyolefin support layer is a non-woven spinlaid polypropylene fabric with hydrophilic pendant groups.

14. The oxygen-sensitive probe of claim 13 wherein the pendant groups are acrylic acid groups.

15. The oxygen-sensitive probe of claim 1 wherein the support layer is between 30 μm and 500 μm thick.

16. The oxygen-sensitive probe of claim 3 wherein the solid state composition is applied only to the first major surface of the support layer.

17. The oxygen-sensitive probe of claim 1 wherein the probe has a change of luminescence lifetime of less than 5% with a change in relative humidity of an analyte gas from 1% to 90%.

18-27. (canceled)
